(19) United States
(12) Patent Application Publication
(10) Pub. No.: US 2009/0142458 A1
(43) Pub. Date: Jun. 4, 2009

(54) PORTABLE ELECTRIC COOLER

(76) Inventor: Kevin McCann, Cincinnati, OH (US)

Correspondence Address: GEORGE R. REARDON, 3356 STATION COURT, LAWRENCVILLE, GA, 30044, US

(21) Appl. No.: 12/325,201

(22) Filed: Nov. 30, 2008

Related U.S. Application Data
(60) Provisional application No. 61/004,966, filed on Nov. 30, 2007.

(51) Int. Cl. A47J 43/00; F25D 23/02; F25B 49/00; A23L 3/28; A23L 1/31; G01N 33/12; F25D 27/00

(52) U.S. Cl. 426/231; 62/498; 62/176.4; 62/264; 99/485; 426/418; 426/248; 426/641

(57) ABSTRACT

The technology described herein provides a device and methods for meat storage, tenderizing, and aging in a temperature and humidity controlled environment. A portable electric cooler is disclosed having first and second sets of low pressure coils coupled to a compressor and three-way solenoid valves in order to alternate in a timed pattern the refrigerant flow in order to allow one set of low pressure coils to defrost while the other set circulates the refrigerant. The cooler includes a temperature sensor, humidity sensor, and humidity controller to monitor and control temperature and humidity levels within the refrigeration chamber. The cooler also includes a pressurized water canister, a spray nozzle, and a pulse solenoid valve. The pulse solenoid valve is configured to discharge a mist through the spray nozzle into the refrigeration chamber as required to maintain a desired humidity.

### CROSS-REFERENCE TO RELATED APPLICATION(S)

The present non-provisional patent application claims the benefit of priority of U.S. Provisional Patent Application No. 61/004,966, which is entitled “COMPACT, PORTABLE, ELECTRIC COOLER FOR DRY AGING BEEF”, which was filed on Nov. 30, 2007, and which is incorporated in full by reference herein.

### FIELD OF THE INVENTION

The technology described herein relates generally to electric cooling devices for food and beverages. More specifically, this technology relates to a device and methods for meat storage, tenderizing, and aging in a temperature and humidity controlled environment.

### BACKGROUND OF THE INVENTION

Many cooling devices are known in the art for cooling food and beverages. By way of example, cooling devices are utilized to store, age, and tenderize meat products in a temperature and humidity controlled environment.

For many, dry aging meat, such as steaks, is a lost art. More tender and flavorful steaks and roasts can be achieved because of a method called dry aging. Typically, capability for the dry-aging of meat is available only to commercial suppliers and to top-tier restaurants. Dry-aging is expensive because of the amount of time required to age the beef and the loss of yield due to dehydration and trimming. Steaks can be dry aged by hanging uncut, just-harvested beef in a refrigerated cooler at a specific temperature and humidity for a predetermined time period, such as for example, 10 to 28 days.

The dry aging process includes exposing beef to open air for up to 28 days in an environment whose temperature, humidity and air circulation is carefully controlled. The dry aging process enhances beef by two means. First, moisture is evaporated from the muscle. This creates a greater concentration of beef flavor and taste. Second, the beef's natural enzymes break down the connective tissue in the muscle, which leads to more tender beef.

Related patents known in the art include the following: U.S. Pat. No. 2,419,119, issued to Christenson on Apr. 15, 1947, discloses an apparatus for treating and storing meat. U.S. Pat. No. 2,494,024, issued to Williams on Jan. 10, 1950, discloses a method and apparatus for refrigerating and aging meat. U.S. Pat. No. 2,816,836, issued to Williams on Dec. 17, 1957, discloses a method for aging meat. U.S. Pat. No. 3,377,941, issued to Jaremus on Apr. 16, 1968, discloses a refrigerator with meat aging and tenderizing compartment. U.S. Pat. No. 3,552,297, issued to Williams on Jan. 5, 1971, discloses an apparatus for aging and flavoring meat. U.S. Pat. No. 4,484,517, issued to Amman on Nov. 27, 1984, discloses a unit for the storage and aging of meat and sausages. U.S. Pat. No. 4,772,480, issued to Yamane on Sep. 20, 1988, discloses a method of controllingly aging edible material. U.S. Pat. No. 5,670,195, issued to Keith on Sep. 23, 1997, discloses a method and apparatus for storing and aging meat. U.S. Pat. No. 6,194,012, issued to Palmer on Feb. 27, 2001, discloses a method for the treatment of meat.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

In various exemplary embodiments, the technology described herein provides a device and methods for meat storage, tenderizing, and aging (wet- and dry-aging) in a temperature and humidity controlled environment.

In one exemplary embodiment, the technology described herein provides a portable electric cooler. The cooler includes a refrigeration chamber, a compressor, the compressor configured to compress and circulate a refrigerant to cool the refrigeration chamber, an expansion device for expanding the refrigerant, a condenser having high pressure coils, a first set of low pressure coils disposed adjacent to the refrigeration chamber, fluidly coupled to the compressor, and configured to hold the refrigerant; and a second set of low pressure coils disposed adjacent to the refrigeration chamber, fluidly coupled to the compressor, and configured to alternatively hold the refrigerant. The first set of low pressure coils and the second set of low pressure coils are alternated with refrigerant flow in order to allow one set of low pressure coils to defrost while the other set circulates the refrigerant.

The cooler also includes a first three-way solenoid valve, the first three-way solenoid valve fluidly disposed after the expansion device and at a beginning of each of the first set of low pressure coils and the second set of low pressure coils and configured to switch between the first set of low pressure coils and the second set of low pressure coils in order to allow one set of low pressure coils to defrost while the other set circulates the refrigerant and a second three-way solenoid valve, the second three-way solenoid valve disposed at an ending of each of the first set of low pressure coils and the second set of low pressure coils and configured to combine two flows back into a single return to the compressor. The first set of low pressure coils and the second set of low pressure coils are maintained at the same volume in order to maintain a stable load on the compressor and to maintain a same cooling capacity.

The cooler further includes a timing device. The first three-way solenoid valve and the second three-way solenoid valve are configured to switch simultaneously on a predetermined time interval as controlled by the timing device.

The cooler further includes a temperature sensor. The refrigeration chamber is configured to be closely maintained at a temperature range immediately above freezing. The temperature is monitored by the temperature sensor and maintained in the range of 33 to 38 degrees Fahrenheit.

The cooler also includes a humidity sensor. The humidity is monitored by the humidity sensor and maintained in the range of 75% to 90% humidity. The cooler further includes a humidity controller, a pressurized water canister, a spray nozzle, and a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity. In at least one embodiment, the pressurized water canister contains flavored water.

The cooler also includes a tinted double-pane glass door disposed upon the refrigeration chamber to allow visibility and monitoring within the refrigeration chamber without opening the door and to disallow unwanted, unfiltered direct light contact with items stored within the refrigeration chamber.

The cooler also includes a UV-C light configured to provide germicidal sterilization within the refrigeration chamber.

The cooler also includes an LED display, to display temperature and humidity levels, an LED interior light to provide illumination within the refrigeration chamber, and a plurality of selection buttons to provide for setting programmatic variables on the portable electric cooler.

In another exemplary embodiment, the technology described herein provides a combined storage, aging, and tenderizing device for meats and beverage cooler. The combined cooling device includes a refrigeration chamber configured to store, age, and tenderize meats and to store and chill beverages, a compressor to compress and circulate a refrigerant to cool the refrigeration chamber, an expansion device to expand the refrigerant, a condenser having high pressure coils, a first set of low pressure coils disposed adjacent to the refrigeration chamber, fluidly coupled to the compressor, and configured to hold the refrigerant, and a second set of low pressure coils disposed adjacent to the refrigeration chamber, fluidly coupled to the compressor, and configured to alternatively hold the refrigerant. The first set of low pressure coils and the second set of low pressure coils are alternated with refrigerant flow in order to allow one set of low pressure coils to defrost while the other set circulates the refrigerant, in order to maintain a narrow temperature range for aging and tenderizing meats. The combined cooling device also includes a first three-way solenoid valve, the first three-way solenoid valve fluidly disposed after the expansion device and at a beginning of each of the first set of low pressure coils and the second set of low pressure coils and configured to switch between the first set of low pressure coils and the second set of low pressure coils in order to allow one set of low pressure coils to defrost while the other set circulates the refrigerant and a second three-way solenoid valve, the second three-way solenoid valve disposed at an ending of each of the first set of low pressure coils and the second set of low pressure coils and configured to combine two flows back into a single return to the compressor.

The combined cooling device includes a temperature sensor to monitor the temperature within the refrigeration chamber. The refrigeration chamber is configured to be closely maintained at a temperature range immediately above freezing for dry-aging meats. The temperature is monitored by the temperature sensor and maintained in the range of 33 to 38 degrees Fahrenheit to keep meats as cold as possible without freezing.

The combined cooling device includes a humidity sensor, wherein the humidity is monitored by the humidity sensor and maintained in the range of 75% to 90% humidity, a humidity controller to control the humidity within the refrigeration chamber conducive to aging meats, a pressurized water canister, a spray nozzle, and a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity for aging meats.

In yet another exemplary embodiment, the technology described herein provides a method for storing, aging, and tenderizing meats. The method include utilizing a refrigeration chamber having a compressor, the compressor configured to compress and circulate a refrigerant to cool the refrigeration chamber, an expansion device for expanding the refrigerant; a condenser having high pressure coils; a first set of low pressure coils disposed adjacent to the refrigeration chamber, fluidly coupled to the compressor, and configured to hold the refrigerant; and a second set of low pressure coils disposed adjacent to the refrigeration chamber, fluidly coupled to the compressor, and configured to alternatively hold the refrigerant. The first set of low pressure coils and the second set of low pressure coils are alternated with refrigerant flow in order to allow one set of low pressure coils to defrost while the other set circulates the refrigerant. The method also includes placing meat into the refrigeration chamber for a predetermined time for aging and tenderizing.

The method further includes utilizing a first three-way solenoid valve, the first three-way solenoid valve fluidly disposed after the expansion device and at a beginning of each of the first set of low pressure coils and the second set of low pressure coils and switching between the first set of low pressure coils and the second set of low pressure coils in order to allow one set of low pressure coils to defrost while the other set circulates the refrigerant and utilizing a second three-way solenoid valve, the second three-way solenoid valve disposed at an ending of each of the first set of low pressure coils and the second set of low pressure coils and combining two flows back into a single return to the compressor, thereby maintaining a narrow temperature range within the refrigeration chamber which is necessary for aging meats.

The method also includes utilizing a temperature sensor to monitor the temperature within the refrigeration chamber and monitoring the temperature by the temperature sensor and adjusting and maintaining a temperature in the range of 33 to 38 degrees Fahrenheit to keep meats cold and moist without freezing.

The method further includes utilizing a humidity sensor, monitoring the humidity by the humidity sensor and maintaining humidity in the range of 75% to 90% humidity, utilizing a humidity controller, controlling the humidity within the refrigeration chamber at a humidity level conducive to aging meats, utilizing a pressurized water canister, utilizing a spray nozzle, utilizing a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity for aging meats, and spraying mist into the refrigeration chamber as needed to maintain a desired humidity for aging meats.

The method also includes utilizing a UV-C light configured to provide germicidal sterilization within the refrigeration chamber and maintaining a sterile environment within the refrigeration chamber.

The method still further includes utilizing flavored water in the pressurized water canister and flavoring the meats with the flavored water.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a front perspective view of a portable electric cooler, according to an embodiment of the technology described herein;

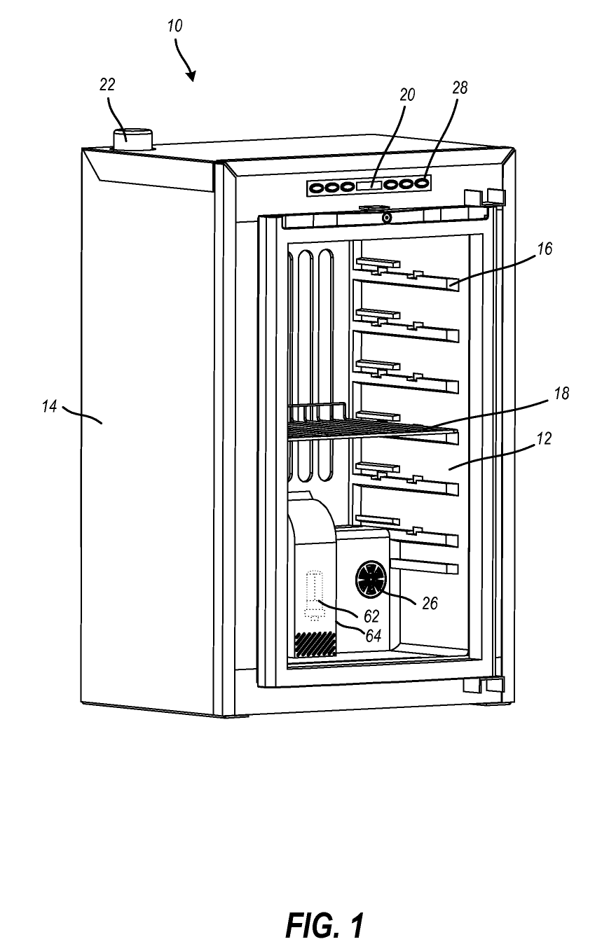

FIG. 2 is an expanded front perspective view of the portable electric cooler of FIG. 1;

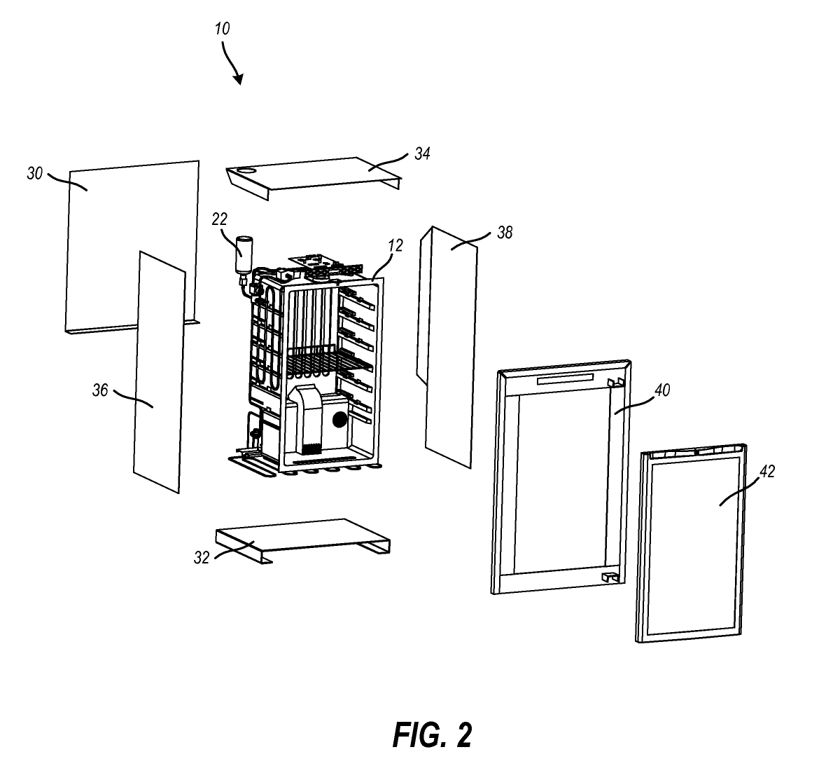

FIG. 3 is a front perspective view of the internal components of the portable electric cooler of FIG. 1, illustrating, in particular, multiple sets of coils controlled by a timer;

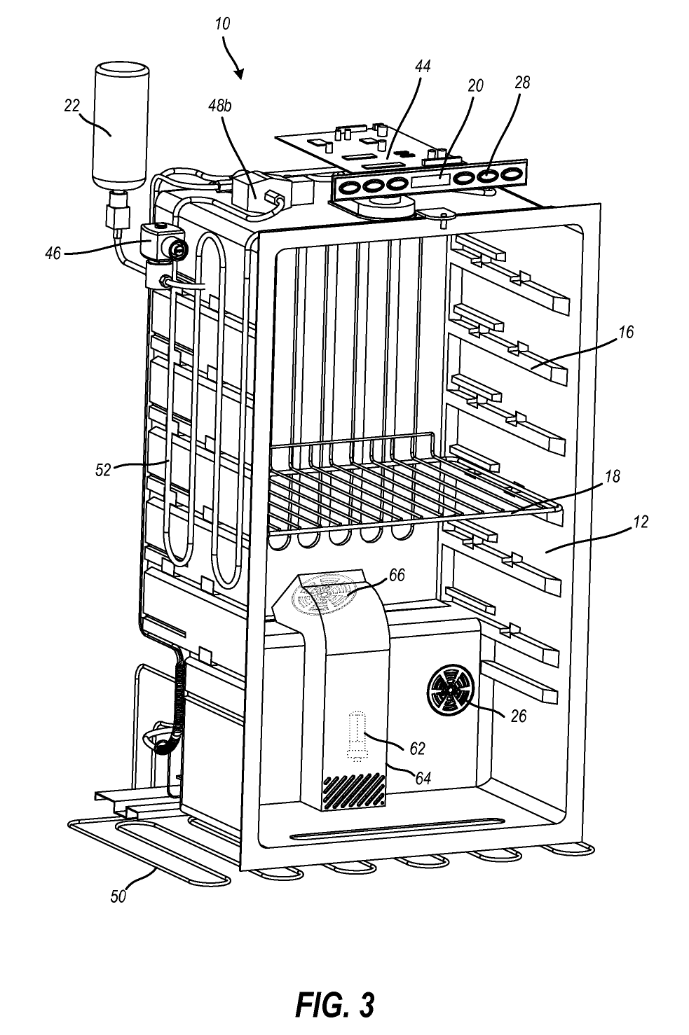

FIG. 4 is a rear perspective view of the internal components of the portable electric cooler of FIG. 1;

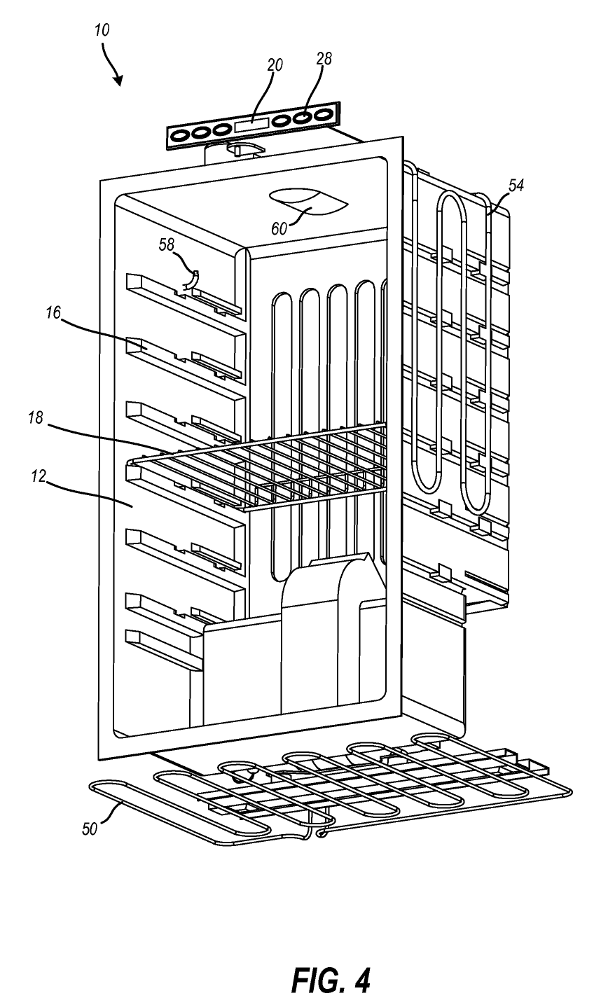

FIG. 5 is a front expanded perspective view of the internal components of the portable electric cooler of FIG. 1;

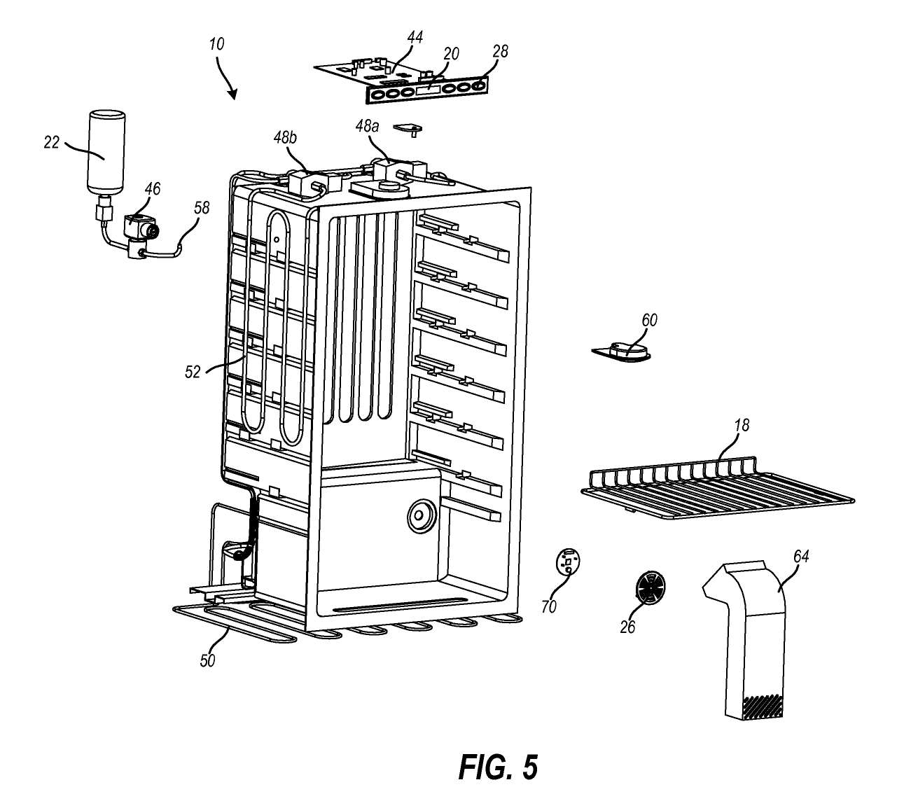

FIG. 6 is front expanded perspective view of the internal components of the portable electric cooler of FIG. 1, illustrating, in particular, multiple sets of coils and a compressor; and

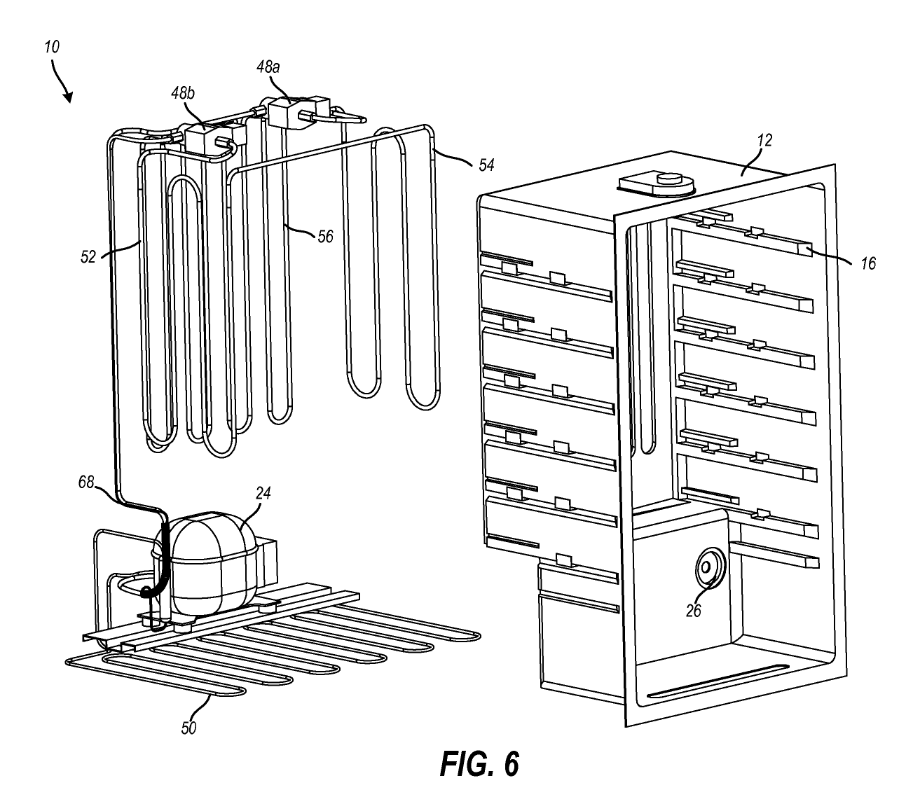

FIG. 7 is a rear expanded perspective view of the internal components of the portable electric cooler of FIG. 1, illustrating, in particular, multiple sets of coils and a compressor.

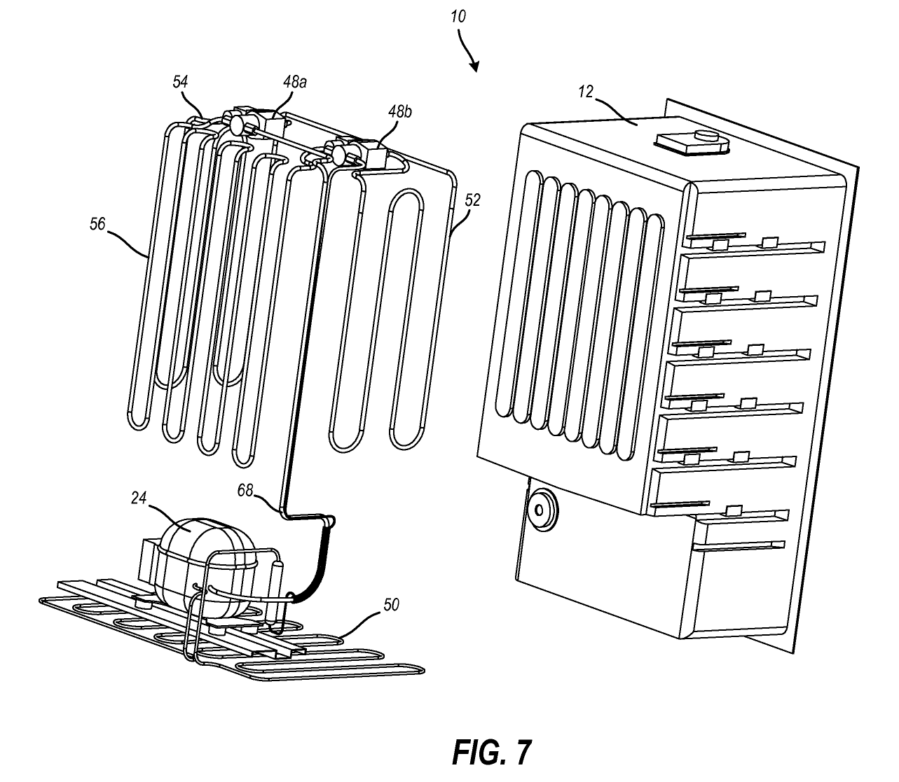

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides a device and methods for meat storage, tenderizing, and aging in a temperature and humidity controlled environment.

Referring now to the Figures, a cooler 10 is depicted. Cooler 10 is a portable, electric cooler that is suitable for home use for storing, aging, and tenderizing meats and cooling beverages such as wine. Cooler 10 includes an inner shell 12 and an outer shell 14. The inner shell 12 can be manufactured from a durable plastic material to form the inside of the refrigeration chamber for storing items such as meats for aging and wines for chilling. The inner shell 12 can be vacuum formed. The outer shell 14 can be manufactured from a durable metal. Disposed between inner shell 12 and outer shell 14 is substantial insulation foam (not shown), such as, for example, approximately double the amount found in conventional refrigeration devices, to help maintain the narrow temperature range required for aging meats. In one embodiment, the cooler 10 is a small portable cooler of approximately three to five cubic feet in interior storage space. This cooler size is suitable for home owners who desire aged steaks, for example, but who do not have access to expensive commercial meat coolers or top-tier restaurants having such.

Outer shell 14 includes a base 32, top 34, rear panel 30, left side panel 36, and right side panel 38. Outer shell 14 includes door frame 40 in which glass door 42 is placed. The glass door 42 is mounted to the door frame 40 such that it can swivel to either open or close as desired. The glass door 42 is tinted in at least one embodiment. A tinted glass door 42 allows visibility and monitoring within the refrigeration chamber without opening the door and disallows unwanted, unfiltered direct light contact with items stored within the refrigeration chamber. Together these components form the outer shell 14 and fully enclose the inner shell 12.

Cooler 10 utilizes a multiplicity of rack mounts 16 to provide various locations within the refrigeration chamber to place the rack 18. Meats can be hung from within the refrigeration chamber or placed on one or more of the racks 18. Cooler 10 also includes a circulation fan 26.

Cooler 10 utilizes a display 20 for the temperature and humidity levels measured within the refrigeration chamber. Display 20 can be an LED display. Although the temperature and humidity levels within the refrigeration chamber can be automatically and electronically adjusted as necessary, the visual display 20 enables one to instantly know the values within the refrigeration chamber.

Cooler 10 also includes a multiplicity of selection buttons 28 that enable a user of the cooler 10 to set-up, program, monitor, and otherwise adjust cooler settings, such as, but not limited to, temperature, humidity, actual time, scheduled time, etc.

Cooler 10 includes a compressor 24. The compressor 24 is configured to compress and circulate a refrigerant to cool the refrigeration chamber. When compressor 24 is running, it compresses the refrigerant in a low-pressure gaseous state to a high-pressure gas. Cooler 10 includes an expansion device 68 for expanding the refrigerant. Cooler 10 includes a condenser having high pressure coils 50.

Cooler 10 includes a first set of low pressure coils 56 disposed adjacent to the refrigeration chamber on the rear. The first set of low pressure coils 56 are fluidly coupled to the compressor 24 and configured to hold the refrigerant. Cooler 10 also includes a second set of low pressure coils disposed adjacent to the refrigeration chamber on both a left side 52 and a right side 54. The second set of low pressure coils 52, 54 are fluidly coupled to the compressor 24 and configured to hold the refrigerant. The first set of low pressure coils 56 and the second set of low pressure coils 52, 54 are alternated with refrigerant flow in order to allow one set of low pressure coils to defrost while the other set circulates the refrigerant.

Cooler 10 also utilizes a three-way solenoid valve 48a. The first three-way solenoid valve 48a is fluidly disposed after the expansion device 68 and at a beginning of each of the first set of low pressure coils and the second set of low pressure coils and configured to switch between the first set of low pressure coils and the second set of low pressure coils in order to allow one set of low pressure coils to defrost while the other set circulates the refrigerant and a second three-way solenoid valve 48b. The second three-way solenoid valve 48b is disposed at an ending of each of the first set of low pressure coils and the second set of low pressure coils and configured to combine two flows back into a single return to the compressor 24. The first set of low pressure coils and the second set of low pressure coils are maintained at the same volume in order to maintain a stable load on the compressor and to maintain a same cooling capacity.

The cooler 10 further includes a control system 44 having a timing device, among other controls, such as for temperature and humidity. The first three-way solenoid valve 48a and the second three-way solenoid valve 48b are configured to switch simultaneously on a predetermined time interval as controlled by the timing device.

The control system 44 also includes a temperature sensor. The refrigeration chamber is configured to be closely maintained at a temperature range immediately above freezing. The temperature is monitored by the temperature sensor and maintained in the range of 33 to 38 degrees Fahrenheit. This range is preferred for aging beef.

The control system 44 also includes a humidity sensor and a humidity controller. The humidity is monitored by the humidity sensor and maintained in the range of 75% to 90% humidity. This range is preferred for aging beef. Sensor location 70 illustrates one location of the temperature sensor and the humidity sensor. As will be apparent to those in the art, such sensors can be placed in varied locations within the refrigeration chamber.

The cooler 10 includes a sterile, pressurized water canister 22, a pulse solenoid valve 46, and a spray nozzle 58. The pulse solenoid valve 46 is fluidly coupled to the pressurized water canister 46 and the spray nozzle 58. The pressurized water canister 46 contains sterile water. The pulse solenoid valve 46 is electronically coupled to the humidity controller and configured to discharge a fine mist through the spray nozzle 58 into the refrigeration chamber as required to maintain a desired humidity. For example, if the humidity drops below a certain value, the pulse solenoid valve 46 is activated to send mists of water into the refrigeration chamber to maintain the desired humidity. The pressurized water canister is replaceable. In at least one embodiment, the pressurized water canister contains flavored water.

The cooler 10 also includes a UV-C light 62 configured to provide germicidal sterilization within the refrigeration chamber. UV-C light 62 is located with duct 64. UV-C light 62 reduces the likelihood of bacterial growth on the steaks, or the like, stored in the refrigeration chamber. The cooler 10 also includes an interior light 60.

Disposed at a top end of duct 64 is a circulation fan 66. Circulation fan 66 provides ventilation to the refrigeration chamber. The dry aging process utilizes a means of evacuating the moist air from the dehydrating beef, to be evacuated from the unit to ensure a slow movement of fresh air around the beef. By way of example, this can be accomplished by installing a vent inside the unit or utilizing the drain for the coils for the same purpose.

In one alternative embodiment, the cooler 10 is utilized in a combination storage, aging, and tenderizing device for meats and beverage cooler for wines and the like.

In at least one embodiment, the cooler 10 can be manufactured utilizing the following general steps: vacuum form the inner shell 12, bend the copper tubing into the proper shape, join the tubing to the 3 way solenoids 48a, 48b, affix the copper tubing to the inner shell 12, bend the outer shell 14 out of sheet metal, place the inner shell and copper tubing inside the outer shell 14, attach the copper tubing to the compressor 24, attach plastic tubing to the water solenoid 46, route one end of the tubing into the inner shell 12 and the other to the externally mounted pressurized water canister 22, wire all the electronics (thermostat, humidity controller, humidity sensor etc.) to the proper locations, fill the area between the inner and outer shells 12, 14 with insulating foam, and attached the glass door 42 with the proper mounting hardware.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. A portable electric cooler comprising:
a refrigeration chamber;
a compressor, the compressor configured to compress and circulate a refrigerant to cool the refrigeration chamber;
an expansion device for expanding the refrigerant;
a condenser having high pressure coils;
a first set of low pressure coils disposed adjacent to the refrigeration chamber, fluidly coupled to the compressor, and configured to hold the refrigerant; and
a second set of low pressure coils disposed adjacent to the refrigeration chamber, fluidly coupled to the compressor, and configured to alternatively hold the refrigerant; and
wherein the first set of low pressure coils and the second set of low pressure coils are alternated with refrigerant flow in order to allow one set of low pressure coils to defrost while the other set circulates the refrigerant.

2. The portable electric cooler of claim 1, further comprising:
a first three-way solenoid valve, the first three-way solenoid valve fluidly disposed after the expansion device and at a beginning of each of the first set of low pressure coils and the second set of low pressure coils and configured to switch between the first set of low pressure coils and the second set of low pressure coils in order to allow one set of low pressure coils to defrost while the other set circulates the refrigerant; and
a second three-way solenoid valve, the second three-way solenoid valve disposed at an ending of each of the first set of low pressure coils and the second set of low pressure coils and configured to combine two flows back into a single return to the compressor.

3. The portable electric cooler of claim 2, further comprising:
a timing device;
wherein the first three-way solenoid valve and the second three-way solenoid valve are configured to switch simultaneously on a predetermined time interval as controlled by the timing device.

4. The portable electric cooler of claim 1, wherein the first set of low pressure coils and the second set of low pressure coils are maintained at the same volume in order to maintain a stable load on the compressor and to maintain a same cooling capacity.

5. The portable electric cooler of claim 1, further comprising:
a temperature sensor;
wherein the refrigeration chamber is configured to be closely maintained at a temperature range immediately above freezing;
wherein the temperature is monitored by the temperature sensor and maintained in the range of 33 to 38 degrees Fahrenheit.

6. The portable electric cooler of claim 1, further comprising:
a humidity sensor;
wherein the humidity is monitored by the humidity sensor and maintained in the range of 75% to 90% humidity.

7. The portable electric cooler of claim 1, further comprising:
a humidity controller;
a pressurized water canister;
a spray nozzle; and
a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity.

8. The portable electric cooler of claim 1, further comprising:
a tinted double-pane glass door disposed upon the refrigeration chamber to allow visibility and monitoring within the refrigeration chamber without opening the door and to disallow unwanted, unfiltered direct light contact with items stored within the refrigeration chamber.

9. The portable electric cooler of claim 1, further comprising:
a UV-C light configured to provide germicidal sterilization within the refrigeration chamber.

10. The portable electric cooler of claim 1, further comprising:
an LED display, to display temperature and humidity levels;
an LED interior light to provide illumination within the refrigeration chamber; and
a plurality of selection buttons to provide for setting programmatic variables on the portable electric cooler.

11. The portable electric cooler of claim 7, wherein the pressurized water canister contains a flavored water.

12. A combined storage, aging, and tenderizing device for meats and beverage cooler, the device comprising:
a refrigeration chamber configured to store, age, and tenderize meats and to store and chill beverages;
a compressor, the compressor configured to compress and circulate a refrigerant to cool the refrigeration chamber;
an expansion device for expanding the refrigerant;
a condenser having high pressure coils;
a first set of low pressure coils disposed adjacent to the refrigeration chamber, fluidly coupled to the compressor, and configured to hold the refrigerant;
a second set of low pressure coils disposed adjacent to the refrigeration chamber, fluidly coupled to the compressor, and configured to alternatively hold the refrigerant;
wherein the first set of low pressure coils and the second set of low pressure coils are alternated with refrigerant flow in order to allow one set of low pressure coils to defrost while the other set circulates the refrigerant, in order to maintain a narrow temperature range for aging and tenderizing meats;
a first three-way solenoid valve, the first three-way solenoid valve fluidly disposed after the expansion device and at a beginning of each of the first set of low pressure coils and the second set of low pressure coils and configured to switch between the first set of low pressure coils and the second set of low pressure coils in order to allow one set of low pressure coils to defrost while the other set circulates the refrigerant; and
a second three-way solenoid valve, the second three-way solenoid valve disposed at an ending of each of the first set of low pressure coils and the second set of low pressure coils and configured to combine two flows back into a single return to the compressor.

13. The combined storage, aging, and tenderizing device for meats and beverage cooler of claim 12, further comprising:
a temperature sensor to monitor the temperature within the refrigeration chamber;
wherein the refrigeration chamber is configured to be closely maintained at a temperature range immediately above freezing for dry-aging meats;
wherein the temperature is monitored by the temperature sensor and maintained in the range of 33 to 38 degrees Fahrenheit to keep meats as cold as possible without freezing.

14. The combined storage, aging, and tenderizing device for meats and beverage cooler of claim 12, further comprising:
a humidity sensor, wherein the humidity is monitored by the humidity sensor and maintained in the range of 75% to 90% humidity.
a humidity controller to control the humidity within the refrigeration chamber conducive to aging meats;
a pressurized water canister;
a spray nozzle; and
a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity for aging meats.

15. A method for storing, aging, and tenderizing meats, the method comprising:
utilizing a refrigeration chamber having a compressor, the compressor configured to compress and circulate a refrigerant to cool the refrigeration chamber; an expansion device for expanding the refrigerant; a condenser having high pressure coils; a first set of low pressure coils disposed adjacent to the refrigeration chamber, fluidly coupled to the compressor, and configured to hold the refrigerant; and a second set of low pressure coils disposed adjacent to the refrigeration chamber, fluidly coupled to the compressor, and configured to alternatively hold the refrigerant; wherein the first set of low pressure coils and the second set of low pressure coils are alternated with refrigerant flow in order to allow one set of low pressure coils to defrost while the other set circulates the refrigerant;
placing meat into the refrigeration chamber for a predetermined time for aging and tenderizing.

16. The method for storing, aging, and tenderizing meats of claim 15, further comprising:
utilizing a first three-way solenoid valve, the first three-way solenoid valve fluidly disposed after the expansion device and at a beginning of each of the first set of low pressure coils and the second set of low pressure coils and switching between the first set of low pressure coils and the second set of low pressure coils in order to allow one set of low pressure coils to defrost while the other set circulates the refrigerant; and
utilizing a second three-way solenoid valve, the second three-way solenoid valve disposed at an ending of each of the first set of low pressure coils and the second set of low pressure coils and combining two flows back into a single return to the compressor;
thereby maintaining a narrow temperature range within the refrigeration chamber which is necessary for aging meats.

17. The method for storing, aging, and tenderizing meats of claim 15, further comprising:
utilizing a temperature sensor to monitor the temperature within the refrigeration chamber; and
monitoring the temperature by the temperature sensor and adjusting and maintaining a temperature in the range of 33 to 38 degrees Fahrenheit to keep meats cold and moist without freezing.

18. The method for storing, aging, and tenderizing meats of claim 15, further comprising:
utilizing a humidity sensor;
monitoring the humidity by the humidity sensor and maintaining humidity in the range of 75% to 90% humidity;
utilizing a humidity controller;
controlling the humidity within the refrigeration chamber at a humidity level conducive to aging meats;
utilizing a pressurized water canister;
utilizing a spray nozzle;
utilizing a pulse solenoid valve, the pulse solenoid valve fluidly coupled to the pressurized water canister and the spray nozzle and electronically coupled to the humidity controller and configured to discharge a mist through the spray nozzle into the refrigeration chamber as required to maintain a desired humidity for aging meats; and
spraying mist into the refrigeration chamber as needed to maintain a desired humidity for aging meats.

19. The method for storing, aging, and tenderizing meats of claim 15, further comprising:
utilizing a UV-C light configured to provide germicidal sterilization within the refrigeration chamber; and
maintaining a sterile environment within the refrigeration chamber.

20. The method for storing, aging, and tenderizing meats of claim 18, further comprising:
utilizing a flavored water in the pressurized water canister; and
flavoring the meats with the flavored water.
